(19) United States
(12) Patent Application Publication
(10) Pub. No.: US 2008/0032409 A1
(43) Pub. Date: Feb. 7, 2008

(54) FLUORESCENT BIOMOLECULE LABELING REAGENTS

(75) Inventors: Jay R. Winkler, Pasadena, CA (US); Jennifer C. Lee, Bethesda, MD (US); Seth B. Harkins, Bethesda, MD (US); Harry B. Gray, Pasadena, CA (US)

Correspondence Address: DAVIS WRIGHT TREMAINE LLP/Los Angeles, 865 FIGUEROA STREET, SUITE 2400, LOS ANGELES, CA, 90017-2566, US

(73) Assignee: California Institute of Technology, Pasadena, CA (US)

(21) Appl. No.: 11/834,861

(22) Filed: Aug. 7, 2007

Related U.S. Application Data
(60) Provisional application No. 60/836,312, filed on Aug. 7, 2006.

(51) Int. Cl. G01N 33/68; C07C 227/00; C07C 229/28; C07C 229/52; C07C 233/64; C07C 381/00

(52) U.S. Cl. 436/86; 560/18; 560/19; 560/49; 562/426; 562/433; 564/155

(57) ABSTRACT

The invention describes fluorescent biomolecule labeling reagents (I-SHark and phI-SHark) and their model compounds. Further described are methods of preparing and using the same.

### PRIORITY INFORMATION

This application claims benefit of priority from U.S. provisional application Ser. No. 60/836,312 filed on Aug. 7, 2006.

### GOVERNMENT RIGHTS

The U.S. Government has certain rights in this invention pursuant to Grant No. GM068461 awarded by the National Institutes of Health.

### FIELD OF INVENTION

This invention relates to fluorescent biomolecule labeling reagents.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Parkinson's disease (PD) is considered a misfolding disease because self-association and aggregation of proteins cause subsequent conformational changes of initially correctly folded proteins. It is a brain disorder that occurs when neurons in the substantia nigra region of the brain die or become impaired. Normally, these cells produce dopamine, which allows for smooth and coordinated function of the body's muscles. The loss of dopamine production in the brain causes the primary symptoms of PD, which include tremors, slow movement, stiffness, and difficulty with balance. Lewy bodies are also found in the surviving neurons in the substantia nigra region of PD patients. Lewy bodies are abnormal protein aggregates with mostly the protein α-syn.

Because the physiological function of α-syn is not yet known, it is very important to understand how its behavior can have an effect on PD. Two mutations in the amino acid sequence of the protein (A53T and A30P) have been linked to early-onset familial PD. This suggests that there is a link between α-syn misfolding and PD, and that high concentrations of α-syn can lead to pathenogenesis. However, the identity of the causative neurotoxic species remains unknown. There is also evidence suggesting that soluble α-syn oligomers are the key cytotoxic agents, rather than insoluble amyloid deposits.

The usual tools used in determining protein structure, such as x-ray crystallography and NMR spectroscopy, provide little insight into the structure of these soluble oligomers (Duus, J. Ø. (1998) Fluorescence energy-transfer probes of conformation in peptides: The 2-aminobenzamide/nitrotyrosine pair. J. Phys. Chem. B 102, 6413-6418). Measurements of fluorescence energy-transfer (FET) kinetics can provide site-specific information about the structure, dynamics, degree of aggregation, and fibrillogenesis mechanisms of amyloid forming proteins (Lee et al. (2004) α-synuclein structures from fluorescence energy-transfer kinetics: Implications for the role of the protein in Parkinson's disease. Proc. Natl. Acad. Sci. 101, 16466-16471). This powerful tool can be employed to obtain a distribution of distances between a fluorescent donor (D) and an energy acceptor (A) (Lyubovitsky et al. (2002) Structural features of the cytochrome c molten globule revealed by fluorescence energy transfer kinetics. J. Am. Chem. Soc. 124, 14840-14841; Lee et al. (2002) Structural features of cytochrome c′ folding intermediates revealed by fluorescence energy-transfer kinetics. Proc. Natl. Acad. Sci. 99, 14778-14782; Wu et al. (1994) Resonance energy transfer: Methods and applications. Anal. Biochem. 218, 1-13; Navon et al. (2001) Distributions of intramolecular distances in the reduced and denatured states of bovine pancreatic ribonuclease A. Folding initiation structures in the C-terminal portions of the reduced protein. Biochemistry 40, 105-118; Pletneva et al. (2005) Nature of the cytochrome c molten globule. J. Am. Chem. Soc. 127, 15370-15371), as the dipole-dipole energy transfer rates are inversely proportional to the sixth power of the fluorescent donor-acceptor distance (Förster, T. (1948) Zwischenmolekulare Energiewanderung und Fluoreszenz. Ann. Phys.-Berlin 2, 55-75). Hence, this technique may extend the understanding on the mechanism of many misfolding diseases, including Alzheimer's (Phu et al. (2005) Fluorescence resonance energy transfer analysis of apolipoprotein E C-terminal domain and amyloid beta peptide (1-42) interaction. J. Neurosci. Res. 80, 877-886) and Parkinson's diseases (Winkler et al. (2006) a-synuclein structures probed by 5-fluorotryptophan fluorescence and F-19 NMR spectroscopy. J. Phys. Chem. B 110, 7058-7061).

To successfully extract distance information between a fluorescent donor and an acceptor, Förster distance (R₀) is an important factor to be considered. Förster distance (Equation 1) is dependent on the orientation factor of the transition dipoles of donor and acceptor (κ²), the refractive index of the surrounding medium (n), the donor fluorescent quantum yield (Φ_{D}), the normalized donor fluorescence spectrum (F_{D}), the acceptor molar absorption spectrum (ε_{A}), and the wavelength (λ).

$${R}_{0}^{6} = 8.785 \times {10}^{- 5} \frac{{κ}^{2}}{{n}^{4}} {Φ}_{D} \int {F}_{D} ( λ ) {ɛ}_{A} ( λ ) {λ}^{4} d λ \qquad ( Equation 1 )$$

Currently, tryptophan and 3-nitrotyrosine have been used as the fluorescent donor and energy acceptor, respectively, to characterize the monomeric structure of α-syn (Lee et al. (2004) α-synuclein structures from fluorescence energy-transfer kinetics: Implications for the role of the protein in Parkinson's disease. Proc. Natl. Acad. Sci. 101, 16466-16471). However, the Förster distance between tryptophan and 3-nitrotyrosine may be too short to measure the distances for aggregation studies (Callis, P. R. (2004) Quantitative prediction of fluorescence quantum yields for tryptophan in proteins. J. Phys. Chem. B 108, 4248-4259). Therefore, there is a need to develop a fluorescent label with a higher quantum yield. The biexponential fluorescence decay of tryptophan (Szabo et al. (1980) Fluorescence decay of tryptophan conformers in aqueous solution. J. Am. Chem. Soc. 102, 554-563) also complicates data interpretation. Therefore, it is preferred to develop FET labels with a single exponential decay.

It is particularly important that the fluorescent label is small enough to not interfere with the native protein structure, and also allow for the maximal rotational freedom of the attached probes (Duus, J. Ø. (1998) Fluorescence energy-transfer probes of conformation in peptides: The 2-aminobenzamide/nitrotyrosine pair. J. Phys. Chem. B 102, 6413-6418). Other ideal properties of FET labels for protein folding studies include a site-specific labeling reaction and an easy purification protocol for the labeled protein.

Fluorescent labeling reagents for biological macromolecules have a wide variety of biotechnology applications. Two key requirements for these reagents are high intrinsic fluorescence and a reactive functional group for conjugation to the biomolecule. Derivatives of 2-amino benzoic acid (Abz) are excellent fluorophores for many biotechnology applications; they are small, hydrophilic, and have high luminescence quantum yields. For protein labeling, thiol-selective functional groups are attractive, owing their ability to target Cys residues. Two thiol-reactive Abz derivatives have been described by Pouchnik et al. (Analytical Biochemistry 1996, 235(1), 26-35). However, these fluorescent molecules have undesirably long tethers between the fluorophore and the biomolecule.

There exists a need in the art for improved molecules for use as labeling reagents. Especially in need are molecules with the following properties: (1) high quantum yield; (2) single exponential decay; (3) minimal interference with native protein structures and high rotational freedom of the attached probes; (4) site-specific labeling reaction; and (5) easy purification protocol for the labeled protein.

### SUMMARY OF THE INVENTION

The following embodiments and aspects thereof are described and illustrated in conjunction with compositions and methods which are meant to be exemplary and illustrative, not limiting in scope.

The inventors have developed several new thiol-specific labeling dyes based on the Abz chromophore.

Specifically, the present invention relates to those fluorescent labels of the Formulas I-VI,

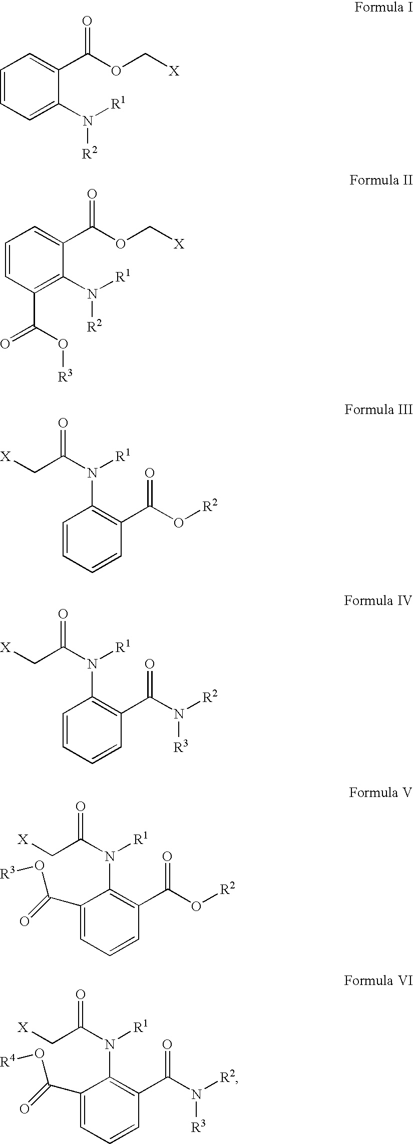

wherein: R¹, R², R³ and R⁴ are each independently selected from hydrogen and functional groups C₂-C₁₈ alkenyl, C₂-C₁₈ alkynyl, C₁-C₁₈ alkyl, aryl, C₁-C₁₈ carboxylate, C₂-C₁₈ alkoxy, C₂-C₁₈ alkenyloxy, C₂-C₁₈ alkynyloxy, aryloxy, C₂-C₁₈ alkoxycarbonyl, C₁-C₁₈ alkylthio, C₁-C₁₈ alkylsulfonyl or C₁-C₁₈ alkylsulfinyl; each optionally substituted with C₁-C₅ alkyl, a halogen, C₁-C₅ alkoxy or with a phenyl group optionally substituted with a halogen, C₁-C₅ alkyl or C₁-C₅ alkoxy; X is selected from F, Cl, Br, I, —SO₃X, a carboxylic acid, a salt of carboxylic acid, CN, nitro, hydroxy, azido, amino, hydrazino, or X is C₁-C₁₈ alkyl, C₁-C₁₈ alkoxy, C₁-C₁₈ alkylthio, C₁-C₁₈ alkanoylamino, C₁-C₁₈ alkylaminocarbonyl, C₂-C₃₆ dialkylaminocarbonyl, C₁-C₁₈ alkyloxycarbonyl, or C₆-C₁₈ arylcarboxamido, the alkyl or aryl portions of which are optionally substituted one or more times by F, Cl, Br, I, hydroxy, carboxylic acid, a salt of carboxylic acid, a carboxylic acid ester of a C₁-C₆ alcohol, —SO₃X, amino, alkylamino, dialkylamino or alkoxy, the alkyl portions of these substituents in turn having 1-6 carbons.

These dyes react with biomolecules via displacement of the reactive atom of X by the sulfur atom of the biomolecule; for example, the sulfur atom on residues (e.g., Cys) on proteins or peptides.

Compounds 1-6 are particularly useful structures of the thiol-selective biomolecule labeling dyes and are shown below.

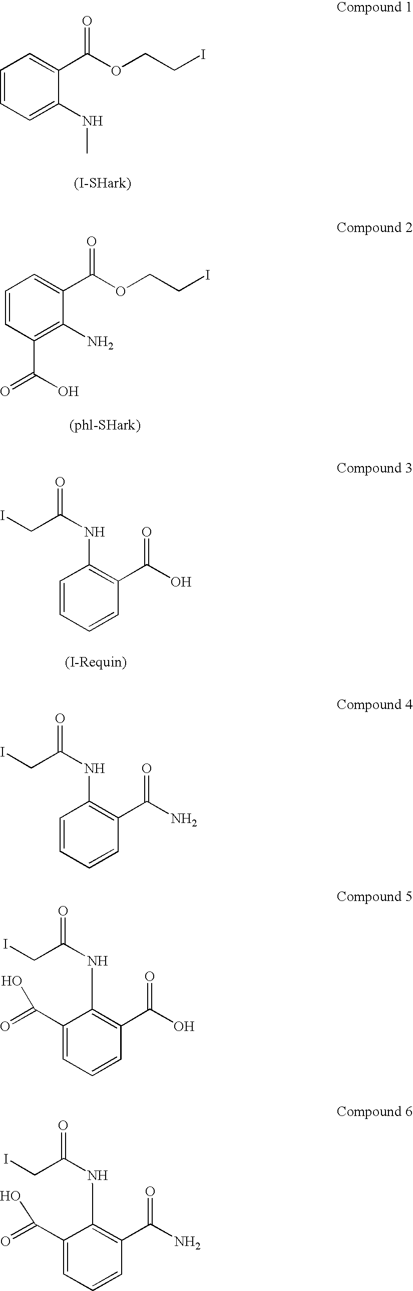

These dyes react with biomolecules via displacement of the iodide from the terminal carbon atom by the sulfur atom of residues (e.g., Cys) on proteins or peptides. In test studies, dyes of Compounds 1 and 2 have been shown to react with the Cys102 residue of Saccharomyces cerevisiae cytochrome c. Early version of the dyes had Bromine (Br) atoms instead of Iodine (I) atoms, but these were found to be less reactive toward sulfhydryl groups. However, versions with Br atoms may still be used in accordance with various embodiments of the present invention.

The present invention also describes model compounds of Formula VII-XII comprising the fluorescent dyes of Formulas I-VI as shown below. The model compounds may be useful in FET technology as these compounds may serve as standard for comparison; that is, they may serve as the control compound for the experiments.

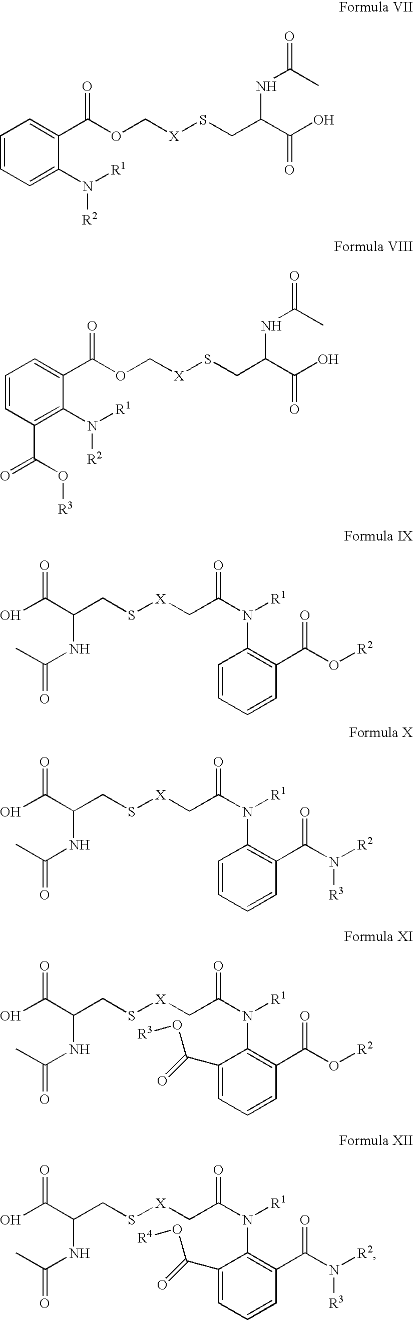

wherein: R¹, R², R³ and R⁴ are each independently selected from hydrogen and functional groups C₂-C₁₈ alkenyl, C₂-C₁₈ alkynyl, C₁-C₁₈ alkyl, aryl, C₁-C₁₈ carboxylate, C₂-C₁₈ alkoxy, C₂-C₁₈ alkenyloxy, C₂-C₁₈ alkynyloxy, aryloxy, C₂-C₁₈ alkoxycarbonyl, C₁-C₁₈ alkylthio, C₁-C₁₈ alkylsulfonyl or C₁-C₁₈ alkylsulfinyl; each optionally substituted with C₁-C₅ alkyl, a halogen, C₁-C₅ alkoxy or with a phenyl group optionally substituted with a halogen, C₁-C₅ alkyl or C₁-C₅ alkoxy; X is selected from F, Cl, Br, I, —SO₃X, a carboxylic acid, a salt of carboxylic acid, CN, nitro, hydroxy, azido, amino, hydrazino, or X is C₁-C₁₈ alkyl, C₁-C₁₈ alkoxy, C₁-C₁₈ alkylthio, C₁-C₁₈ alkanoylamino, C₁-C₁₈ alkylaminocarbonyl, C₂-C₃₆ dialkylaminocarbonyl, C₁-C₁₈ alkyloxycarbonyl, or C₆-C₁₈ arylcarboxamido, the alkyl or aryl portions of which are optionally substituted one or more times by F, Cl, Br, I, hydroxy, carboxylic acid, a salt of carboxylic acid, a carboxylic acid ester of a C₁-C₆ alcohol, —SO₃X, amino, alkylamino, dialkylamino or alkoxy, the alkyl portions of these substituents in turn having 1-6 carbons.

Compounds 7-12 are particularly useful structures of the model compounds and are shown below.

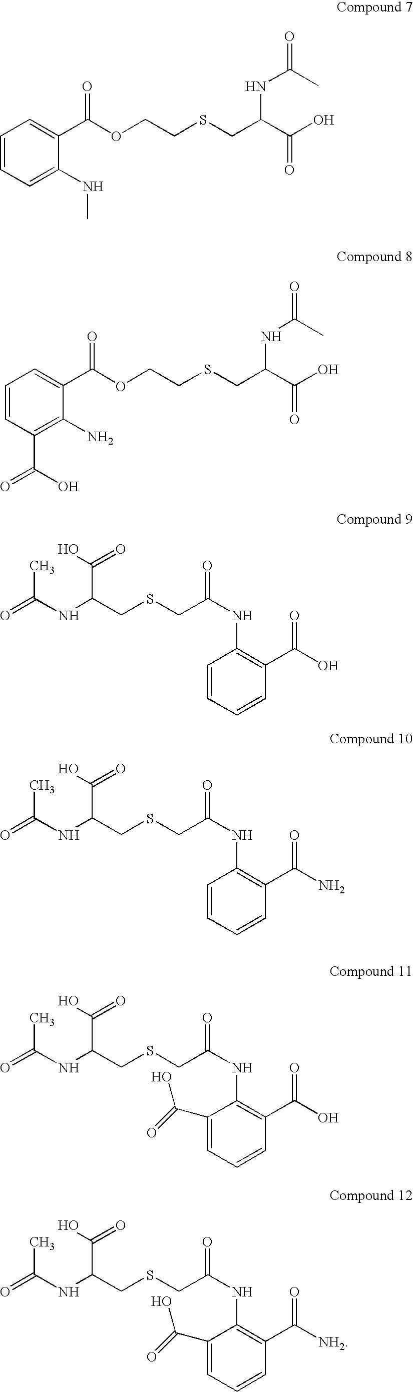

The present invention also describes the synthesis of new fluorescent labels and their model compounds. Further, the present invention provides for a method of labeling biomolecules such as proteins and peptides; specifically, the cysteine or the lysine residue of the protein or peptide. The N-terminus may also be labeled with the fluorescent label of the present invention.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts molar absorption spectra of cyt c, and fluorescence spectra of (A) I-SHark and (B) phI-Shark in accordance with various embodiments of the present invention.

FIG. 2 depicts steady-state fluorescence spectra for 5 μM phI-SHark labeled cyt c in 20 mM NaPᵢ (dotted line) and 6 M GuHCl (solid line), pH 7.4 at 25° C. in accordance with various embodiments of the present invention.

FIG. 3 depicts distribution of fluorescence decay rates in accordance with various embodiments of the present invention. P(k), for 5 μM phI-SHark labeled cyt c in 20 mM NaPᵢ (A) and 6 M GuHCl (B) at pH 7.4 and 25° C., from NNLS analyses of the FET kinetics.

FIG. 4 depicts steady-state fluorescence spectra for 5 μM phI-SHark model complex (4) in 20 mM NaPᵢ (dotted line) and 6 M GuHCl (solid line), pH 7.4 at 25° C., in accordance with various embodiments of the present invention.

FIG. 5 depicts distribution of fluorescence decay rates in accordance with various embodiments of the present invention. P(k), for 5 μM phI-SHark model complex (4) in 20 mM NaPᵢ (A) and 6 M GuHCl (B) at pH 7.4 and 25° C., from NNLS analyses of the FET kinetics.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); and March, Advanced Organic Chemistry Reactions, Mechanisms and Structure 5^{th} ed., J. Wiley & Sons (New York, N.Y. 2001), provide one skilled in the art with a general guide to many of the terms used in the present application.

“Biomolecule” as used herein refers to an organic molecule that is part of a living organism. Examples of biomolecules include, but are not limited to: lipids, hormones, carbohydrates, polysaccharides, amino acids, nucleic acids, peptides, and proteins.

“Room temperature” as used herein refers to temperatures of about 15 to about 30 degrees Celsius.

The present invention describes the synthesis and spectroscopic characterization of new fluorescent labels and their model compounds.

Specifically, an iodine is engineered in the label to undergo S_{N}2 reaction with the sulfhydryl group in the cysteine. As described herein, the position iodine occupies on the fluorescent label may be occupied by a different atom or functional group whereby a covalent linkage between the dye and the biomolecule is achieved.

Cyt c, a well studied protein with a native cysteine residue, was successfully labeled with the new dyes of compound 1 and compound 2 to prove that they may be used as probes for FET studies.

The new labels and their corresponding model compounds (cysteinic conjugates) were successfully synthesized by one-step reactions. The products were purified by either flash column chromatography or crystallization.

When designing the structure of new FET labels, minimal perturbation of the native protein structure and high rotational freedom of the fluorophore should be considered. Therefore, I-SHark (2-iodoethyl 2-(methylamino)benzoate) (Compound 1) and phI-SHark (2-amino-3-[(2-iodoethoxy)carbonyl]benzoic acid) (Compound 2) were designed to cause minimal steric hindrance. Iodoacetamide is frequently used as the reactive site for cysteine labeling. However, iodine was chosen instead in designing these labels to provide higher conformational freedom. The fluorophore conformational freedom was further enhanced by the ethylenic chain linking the cysteine and fluorophore.

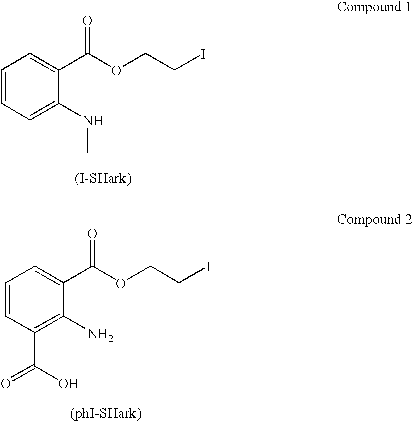

The inventors also synthesized a model compound containing I-SHark (2-(acetylamino)-3-[(2-{[2-(methylamino)benzoyl]oxy}ethyl)thio]propanoic acid) (Compound 7) and a model compound containing phI-SHark (3-[(2-{[2-(acetylamino)-2-carboxyethyl]thio}ethoxy)carbonyl]-2-aminobenzoic acid) (Compound 8).

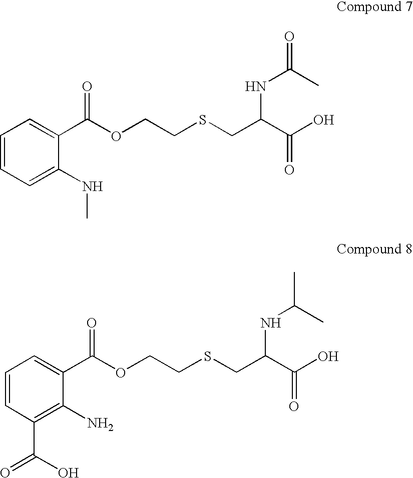

As an example of labeling a protein, the labeling of Cys102 of cyt C was performed. The functionalization of cyt c involves a linker between the fluorescent probe and Cys102 of the protein. However, the thiol of Cys102 is not solvent-exposed enough to react with the labels. Therefore, the protein was unfolded by GuHCl to encourage protein derivatization. TCEP was also added to avoid the oxidation of sulfur groups. The reaction was carried out at a pH=7.6 to where the thiol group was partially deprotonated, while the amine side chain of lysine, which is a main reactive agonist of cysteine, were almost fully protonated. The duration of protein labeling was optimized at 40 h, as protein denaturation and the oxidation of thiolic groups reduce labeling yields at longer reaction times.

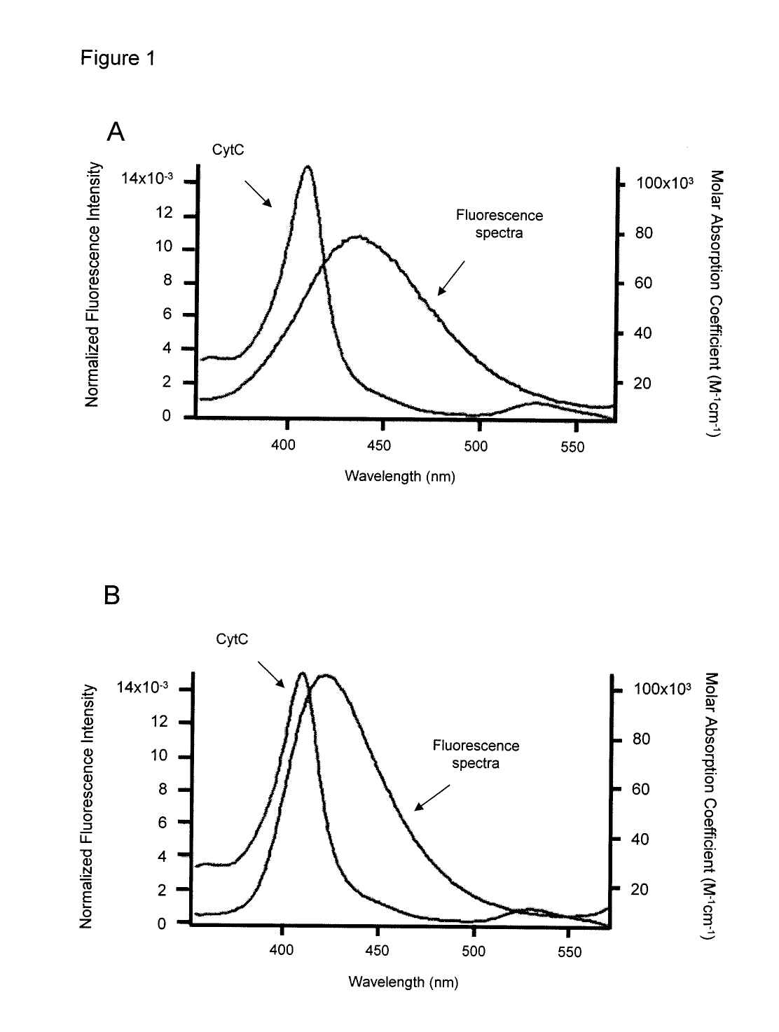

Table 1 shows the fluorescent quantum yields of the labels, calculated overlap integral, and Förster distances between I-SHark/phI-SHark and cyt c heme group. The overlap between the absorption of cyt c and phI-SHark is better than that of cyt c and I-SHark (See FIG. 1). It is also demonstrated that phI-SHark has a higher quantum yield and longer Förster distance with cyt c than I-SHark. One disadvantage of I-SHark is its low water solubility; nonetheless, it may be used in accordance with various embodiments of the present invention. DMSO was added to enhance dye solubility in the labeling reaction. However, the amount of DMSO added (10% v/v) is limited to avoid protein precipitation. On the other hand, the second carboxylic acid group makes phI-SHark more soluble than I-SHark in the aqueous labeling environment. Since phI-SHark has been demonstrated to be a better label in aqueous environments, the following discussion is focused on the spectroscopic characterization of phI-SHark.

TABLE 1
Fluorescent quantum yields of I-SHark and phl-SHark, calculated
overlap integral and Förster distances between
I-SHark/phl-SHark and heme group.

| | Quantum | Förster | Overlap Integral |
|---|---|---|---|
| | Yield | Distance (Å) | (M⁻¹ cm⁻¹ nm⁴) |
| I-SHark | 0.32 | 50 | 8.7 × 10¹⁴ |
| phl-SHark | 0.48 | 60 | 8.1 × 10¹⁵ |

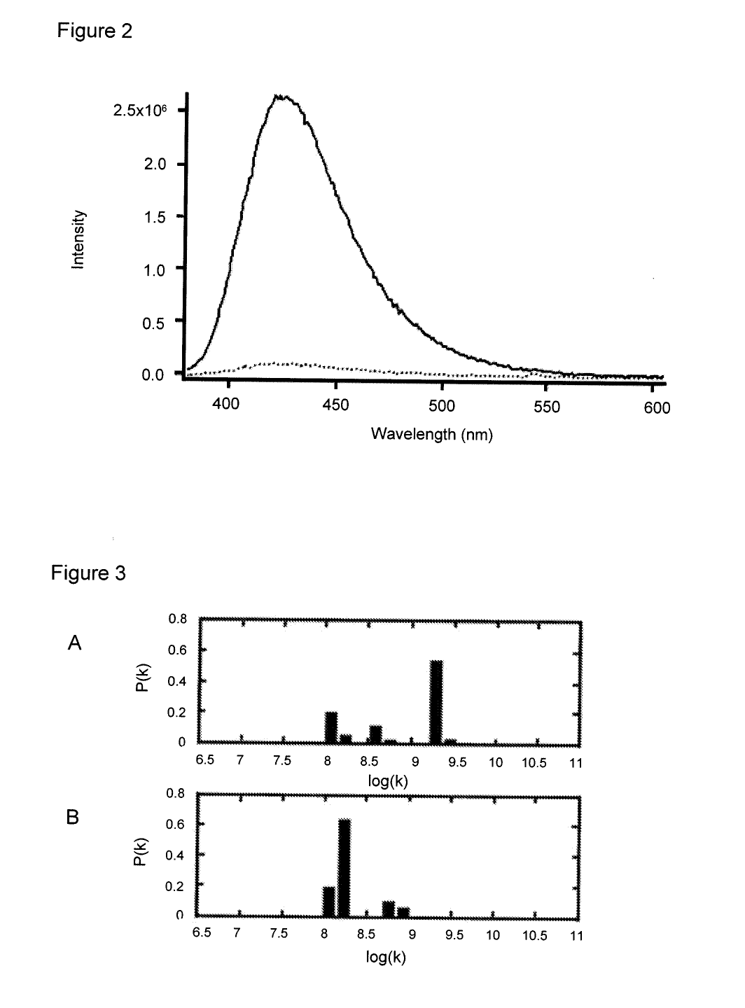

The phI-SHark labeled cyt c is characterized spectroscopically in its native and denatured conformation to illustrate the ability of the label to provide distance information. When the protein is folded, the distance between Cys102 to the iron heme center is 13.9 Å. Therefore, when the protein is folded, the fluorescence emitted by the label should be quenched by the heme group. On the other hand, when the protein is unfolded, the distance between the heme group and fluorophore should be too large for the quenching to be effective. In fact, steady-state fluorescence spectra show a significantly reduced emission for the folded protein compared to the unfolded protein (see FIG. 2). FET kinetics (see FIG. 3) also show a much faster decay for the unfolded protein (6×10⁻⁹ s⁻¹, 80%), when compared to the folded protein (8×10⁻⁹ s⁻¹, 30%, 5×10⁻¹⁰ s⁻¹, 60%).

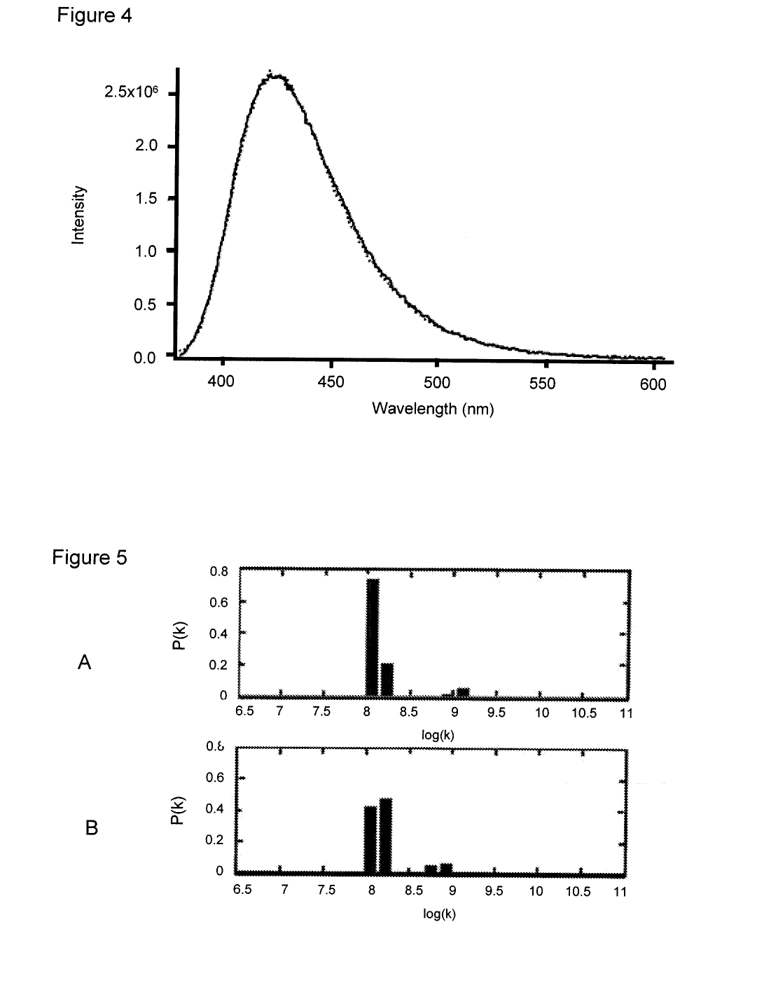

Control experiments using the phI-SHark model complex in NaPᵢ buffer and 6 M GuHCl were conducted. Comparable steady-state fluorescence emissions (see FIG. 4) and time-resolved lifetimes (see FIG. 5) show that phI-SHark behaves similarly in buffer (8×10⁻⁹ s⁻¹, 90%) and high concentration of GuHCl (7×10⁻⁹ s⁻¹, 90%). It can also be noted that the unquenched emission of phI-SHark is singly exponential.

In summary, the highly fluorescent dyes, I-SHark and phI-SHark, were synthesized and characterized. Spectroscopic characterization of phI-SHark labeled cyt c shows that phI-SHark fulfills the requirements needed for an excellent label for FET studies. Its minimal steric hindrance, high rotational freedom, single exponential fluorescent lifetime, high quantum yield, ease of synthesis, and high solubility in water make it attractive to be paired with other energy acceptors to provide distance information during protein folding events.

Both I-SHark and phI-SHark are prepared by ester formation using 2-iodoethanol and a commercially available acid precursor.

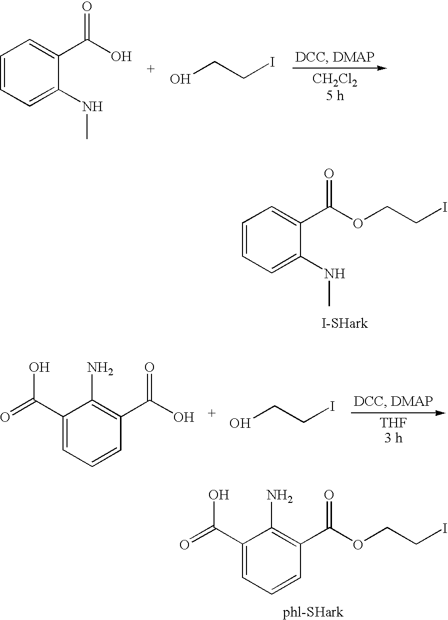

In the case of I-SHark, the precursor is 2-aminobenzoic acid. For phI-SHark, the precursor is 2-amino-isophthalic acid. Ester formation is effected using 4-N,N-dimethylamino-pyridine (DMAP) and dicyclohexyl-carbodiimide (DCC) reagents. Detailed synthetic procedures and analytical data for the dyes and model conjugates are described herein.

I-Requin and its derivatives are prepared as follows.

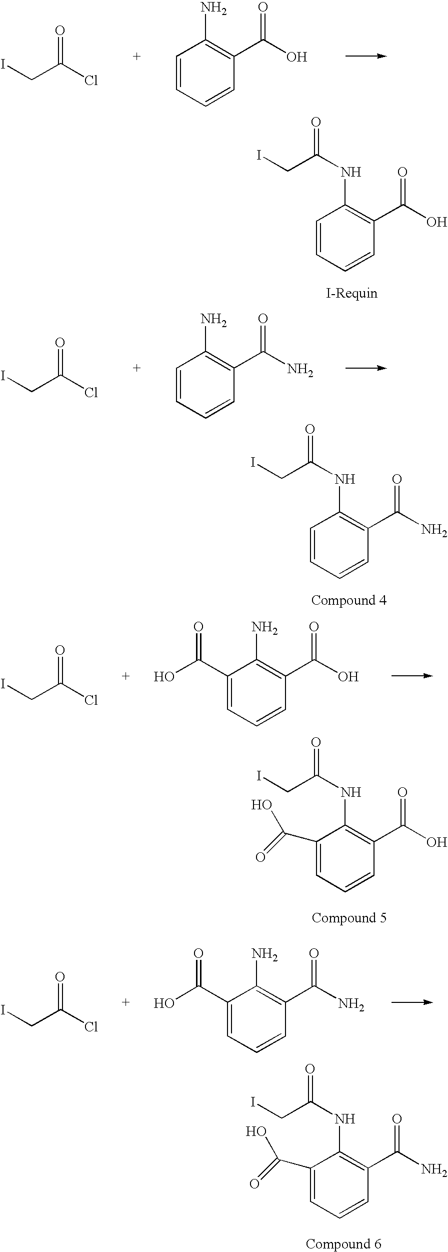

The present invention provides for compositions comprising fluorescent aminobenzoic acid derivatives, methods of synthesizing the compositions, and methods of using the compositions for labeling biomolecules, such as proteins and peptides.

One embodiment of the present invention provides for a fluorescent dye composition comprising a compound selected from the group consisting of Formula I, Formula II, Formula IV, Formula V, Formula VI, salts thereof, and combination thereof, or Formula III in combination with Formulas I, II, IV, V and/or VI, wherein R¹, R², R³ and R⁴ are independently selected from hydrogen and functional groups C₂-C₁₈ alkenyl, C₂-C₁₈ alkynyl, C₁-C₁₈ alkyl, aryl, C₁-C₁₈ carboxylate, C₂-C₁₈ alkoxy, C₂-C₁₈ alkenyloxy, C₂-C₁₈ alkynyloxy, aryloxy, C₂-C₁₈ alkoxycarbonyl, C₁-C₁₈ alkylthio, C₁-C₁₈ alkylsulfonyl or C₁-C₁₈ alkylsulfinyl; each optionally substituted with C₁-C₅ alkyl, a halogen, C₁-C₅ alkoxy or with a phenyl group optionally substituted with a halogen, C₁-C₅ alkyl or C₁-C₅ alkoxy; X is selected from F, Cl, Br, I, —SO₃X, a carboxylic acid, a salt of carboxylic acid, CN, nitro, hydroxy, azido, amino, hydrazino, or X is C₁-C₁₈ alkyl, C₁-C₁₈ alkoxy, C₁-C₁₈ alkylthio, C₁-C₁₈ alkanoylamino, C₁-C₁₈ alkylaminocarbonyl, C₂-C₃₆ dialkylaminocarbonyl, C₁-C₁₈ alkyloxycarbonyl, or C₆-C₁₈ arylcarboxamido, the alkyl or aryl portions of which are optionally substituted one or more times by F, Cl, Br, I, hydroxy, carboxylic acid, a salt of carboxylic acid, a carboxylic acid ester of a C₁-C₆ alcohol, —SO₃X, amino, alkylamino, dialkylamino or alkoxy, the alkyl portions of these substituents in turn having 1-6 carbons.

Moreover, X is meant to be used as a useful covalent linkage between the dye and a biomolecule. Table 2 summarizes examples of such linkages.

TABLE 2

| Electrophilic Group | Nucleophilic Group | Resulting Covalent Linkage |
|---|---|---|
| activated esters* | amines/anilines | carboxamides |
| acrylamides | thiols | thioethers |
| acyl azides** | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| alkyl sulfonates | thiols | thioethers |
| alkyl sulfonates | carboxylic acids | esters |
| alkyl sulfonates | alcohols/phenols | ethers |
| anhydrides | alcohols/phenols | esters |
| anhydrides | amines/anilines | carboxamides |
| aryl halides | thiols | thiophenols |
| aryl halides | amines | aryl amines |
| aziridines | thiols | thioethers |
| boronates | glycols | boronate esters |
| carboxylic acids | amines/anilines | carboxamides |
| carboxylic acids | alcohols | esters |
| carboxylic acids | hydrazines | hydrazides |
| carbodiimides | carboxylic acids | N-acylureas or anhydrides |
| diazoalkanes | carboxylic acids | esters |
| epoxides | thiols | thioethers |
| haloacetamides | thiols | thioethers |
| halotriazines | amines/anilines | aminotriazines |
| halotriazines | alcohols/phenols | triazinyl ethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphite esters |
| silyl halides | alcohols | silyl ethers |
| sulfonate esters | amines/anilines | alkyl amines |
| sulfonate esters | thiols | thioethers |
| sulfonate esters | carboxylic acids | esters |
| sulfonate esters | alcohols | ethers |
| sulfonyl halides | amines/anilines | sulfonamides |
| sulfonyl halides | phenols/alcohols | sulfonate esters |

*Activated esters, as understood in the art, generally have the formula —COΩ, where Ω is a good leaving group (e.g. succinimidyloxy (—OC₄H₄O₂) sulfosuccinimidyloxy (—OC₄H₃O₂—SO₃H), -1-oxybenzotriazolyl (—OC₆H₄N₃); or an aryloxy group or aryloxy substituted one or more times by electron withdrawing substituents such as nitro, fluoro, chloro, cyano, or trifluoromethyl, or combinations thereof, used to form activated aryl esters; or a carboxylic acid activated by a carbodiimide to form an anhydride or mixed anyhydride —OCNR^{a}NHR^{b}, where R^{a} and R^{b}, which may be the same or different, are are C₁-C₆ alkyl, C₁-C₆ perfluoroalkyl, or C₁-C₆ alkoxy; or cyclohexyl, 3-dimethylaminopropyl, or N-morpholinoethyl).
**Acyl azides can also rearrange to isocyanates

In various specific embodiments, the fluorescent dye composition comprises a compound selected from the group consisting of 2-iodoethyl 2-(methylamino)benzoate (“I-SHark”) (Compound 1), 2-amino-3-[(2-iodoethoxy)carbonyl]benzoic acid (“phI-SHark”) (Compound 2), Compound 4, Compound 5, Compound 6, salts thereof and combinations thereof, or Compound 3 in combination with Compounds 1, 2, 4, 5, and/or 6.

In another embodiment, these fluorescent compositions further comprise a biomolecule; particularly, a protein, a peptide, or both. In another embodiment, the fluorescent composition comprising compound 3 further comprises a biomolecule; particularly, a protein, a peptide, or both.

Another embodiment of the present invention provides for fluorescent compositions comprising model compounds selected from the group consisting of Formula VII, Formula VIII, Formula IX, Formula X, Formula XII, Formula XII, salts thereof and combinations thereof.

In various specific embodiments, the compositions comprises model compounds selected from the group consisting of 2-(acetylamino)-3-[(2-{[2-(methylamino)benzoyl]oxy}ethyl)thio]propanoic acid (Compound 7), 3-[(2-[2-(acetylamino)-2-carboxyethyl]thio}ethoxy)carbonyl]-2-aminobenzoic acid (Compound 8), Compound 9, Compound 10, Compound 11, Compound 12, salts thereof and combinations thereof. These model compounds may be useful in technologies involving fluorescence energy-transfer kinetics as they may be used as a control compound.

Another embodiment of the present invention provides for a method of synthesizing I-SHark (2-iodoethyl 2-(methylamino)benzoate) (Compound 1) or a salt thereof, comprising: providing a quantity of 2-(methylamino)-benzoic acid and a quantity of 2-iodoethanol; and reacting the quantity of 2-(methylamino)-benzoic acid and the quantity of 2-iodoethanol to form 2-iodoethyl 2-(methylamino)benzoate or a salt thereof.

In one embodiment, reacting the quantity of 2-(methylamino)-benzoic acid and the quantity of 2-iodoethanol comprises adding a quantity of 4-dimethylaminopyridine (“DMAP”) and a quantity of dicyclohexylcarbodiimide (“DCC”) to the quantities of 2-(methylamino)-benzoic acid and 2-iodoethanol.

In various embodiments, the quantity of 2-(methylamino)-benzoic acid is about 16.9 mmol and the quantity of 2-iodoethanol is about 49.8 mmol. These quantities may be scaled accordingly. In a particular embodiment, the quantity of 2-(methylamino)-benzoic acid and 2-iodoethanol is provided at a ratio of about 1:3. In various embodiments, the quantity of DMAP is about 2.62 mmol and the quantity of DCC is about 18.4 mmol.

In one particular embodiment, the quantity of DMAP is added to the solution before the quantity of DCC is added to the solution. Particularly useful is having the quantity of DMAP, the quantity of 2-(methylamino)-benzoic acid and the quantity of 2-iodoethanol dissolved together prior to the addition of the quantity of DCC. These reagents may be dissolved in methylene chloride (CH₂Cl₂); particularly, in dry CH₂Cl₂. In one embodiment, the quantity of DCC is added dropwise. In another embodiment, the quantity of DCC is in CH₂Cl₂; particularly, dry CH₂Cl₂. Methylene chloride is generally available in wet form (CH₂Cl₂.H₂O) and thus dry methylene chloride as used herein refers to methylene chloride wherein the water component has been removed.

In another embodiment, the method further comprises stirring the solution. In various embodiments, the solution is stirred under Argon (“Ar”), at room temperature and/or for about five hours.

In another embodiment, the method further comprises concentrating the solution, which may be performed by filtering the solution to create a filtrate, washing the filtrate, and/or drying the washed filtrate. In various embodiments, the solution may be filtered through celite and/or washed with CH₂Cl₂ and the washed filtrate may be dried over sodium sulfate (Na₂SO₄).

In another embodiment, the method further comprises purifying the concentrated solution. This may be accomplished by using flash column chromatography to create a purified oil and/or by crystallization. The purified oil may be acidified; for example, with hydrogen chloride (HCl) to create a chloride salt. The chloride salt may be crystallized from methanol/ether. Thus, the crystallized product comprises 2-iodoethyl 2-(methylamino)benzoate or a salt thereof.

In a particular embodiment, the method of synthesizing 2-iodoethyl 2-(methylamino)benzoate or a salt thereof comprises: providing about 16.9 mmol of 2-(methylamino)-benzoic acid, about 2.62 mmol DMAP and about 29.8 mmol of 2-iodoethanol; dissolving the 2-(methylamino)-benzoic acid, DMAP and 2-iodoethanol in dry CH₂Cl₂ creating a solution; adding about 18.4 mmol of DCC in dry CH₂Cl₂ dropwise to the solution; and stirring the solution under Ar at room temperature for about 5 hours. The method may further comprise a step selected from the group consisting of filtering the solution through celite, washing the solution with CH₂Cl₂, drying the solution over Na₂SO₄, concentrating the solution under a vacuum, purifying the concentrated solution under flash column chromatography to produce a purified green oil, acidifying the purified green oil with concentrated HCl to produce a chloride salt, crystallizing the chloride salt from methanol/ether and combinations thereof. In alternative embodiments, the quantities of 2-(methylamino)-benzoic acid, DMAP, 2-iodoethanol and DCC may be scaled accordingly.

Another embodiment of the present invention provides for a method of synthesizing phI-SHark (2-amino-3-[(2-iodoethoxy)carbonyl]benzoic acid) (Compound 2) or a salt thereof, comprising: providing a quantity of 2-iodoethanol, and a quantity of 2-aminoisophthalic acid; and reacting the quantity of 2-iodoethanol and the quantity of 2-aminoisophthalic acid to form phI-SHark (2-amino-3-[(2-iodoethoxy)carbonyl]benzoic acid) (Compound 2) or a salt thereof.

In one embodiment, reacting the quantity of 2-iodoethanol and the quantity of 2-aminoisophthalic acid comprises adding a quantity of DMAP and the quantity of 2-iodoethanol to a stirring solution of 2-aminoisophthalic acid in tetrahydrofuran (“THF”); and adding a quantity of DCC dropwise; particularly, DCC in dry methylene chloride. The method may further comprise stirring the solution for about 4 hours, stirring the solution at room temperature, and/or drying the solution. The solution may be dried under vacuum. The dried residue may be redissolved in diethyl ether and extracted with 5% aqueous sodium bicarbonate (NaHCO₃). For example, three 30 mL aliquots of the aqueous NaHCO₃ may be used to extract the product. One of skill in the art will be able to readily determine the number of aliquots or the quantity in each aliquot that is used to extract the product. Acidification of the aqueous layer to a pH of less than 4 may be performed to recover the 2-aminoisophthalic acid, one of the starting reagents. The organic extracts may be combined and dried over MgSO₄, filtered, and concentrated to yield a pale yellow solid (i.e., the crude product). In one embodiment, the crude product is purified by chromatography. In another embodiment, the purified product is dissolved in methanol and crystallized from methanol/methylene chloride. This may be accomplished by adding a 6M NH₄0H solution.

In one embodiment, the quantity of 2-iodoethanol is about 4.45 mmol. In another embodiment, the quantity of 2-aminoisophthalic acid is about 1.50 mmol. These quantities may be scaled accordingly. In another embodiment, the quantity of 2-aminoisophthalic acid and 2-iodoethanol is provided at a ratio of about 1:3. In another embodiment, the quantity of DMAP is about 0.2 mmol. In another embodiment, the quantity of DCC is about 1.65 mmol.

Another embodiment of the present invention provides for a method of synthesizing I-Requin (Compound 3) or I-Requin derivatives (Compounds 4-6). The method comprises condensation of an amino group on the dye precursor with an iodoacetyl chloride reagent. The condensation reaction is run in a mildly basic aqueous solution to ensure deprotonation of the amine group. Upon the completion of the reaction, the solution is acidified and the product (I-Requin, or I-Requin derivatives) precipitates. Alternatively, isolation of the I-Requin derivatives may be achieved by procedures that are readily known to one of skill in the art. In alternative embodiments, halo-acetyl chloride reagents may be used.

Another embodiment of the present invention provides for a method of synthesizing 2-(acetylamino)-3-[(2-{[2-(methylamino)benzoyl]oxy}ethyl)thio]propanoic acid (Compound 7) or a salt thereof. Compound 7 contains I-SHark and may be useful in technologies involving fluorescence energy-transfer kinetics as it may be used as a control compound. The method of synthesizing 2-(acetylamino)-3-[(2-{[2-(methylamino)benzoyl]oxy}ethyl)thio]propanoic acid comprises: providing a quantity of 2-iodoethyl 2-(methylamino)benzoate (Compound 1) or a salt thereof; and reacting the 2-iodoethyl 2-(methylamino)benzoate or the salt thereof with N-acetyl cysteine. In one embodiment, reacting the iodoethyl 2-(methylamino)benzoate with the N-acetyl-cysteine comprises adding a quantity of 1,8-Diazabicyclo[5.4.0]undec-7-ene (“DBU”) to a stirring solution of iodoethyl 2-(methylamino)benzoate or the salt thereof and N-acetyl cysteine in DMF; particularly, in dry DMF. The DBU may be added dropwise.

In one embodiment, the quantity of 2-iodoethyl 2-(methylamino)benzoate may be about 1.00 mmol. In another embodiment, the quantity of N-Acetyl-cysteine may be about 2.0 mmol. In another embodiment the quantity of 2-iodoethyl 2-(methylamino)benzoate and the quantity of N-Acetyl-cysteine may be provided at a ratio of about 1:2.

In another embodiment, the method further comprises stirring the mixture; for example stirring the mixture under Ar and/or for about 1 hour. In another embodiment, the method further comprises drying the solution to form a dried product; for example, drying the solution under vacuum. In another embodiment, the method further comprises redissolving the dried product in HCl and/or extracting the solution with ethyl acetate. The organic layers may be reduced to dryness under vacuum and redissolved in methanol. An about 30% solution of NH₄OH may be added and then the 2-(acetylamino)-3-[(2-{[2-(methylamino)benzoyl]oxy}ethyl)thio]propanoic acid (Compound 7) or the salt thereof may be crystallized from methanol/ether.

Another embodiment of the present invention provides for a method of synthesizing 3-[(2-{[2-(acetylamino)-2-carboxyethyl]thio}ethoxy)carbonyl]-2-aminobenzoic acid (Compound 8) or a salt thereof. Compound 8 contains phI-SHark and may be useful in technologies involving fluorescence energy-transfer kinetics as it may serve as a control compound. The method of synthesizing 3-[(2-[2-(acetylamino)-2-carboxyethyl]thio}ethoxy)carbonyl]-2-aminobenzoic acid (Compound 8) or a salt thereof comprises providing a quantity of 2-amino-3-[(2-iodoethoxy)carbonyl]benzoic acid or the salt thereof and a quantity of N-Acetyl-cysteine; and reacting the quantity of 2-amino-3-[(2-iodoethoxy)carbonyl]benzoic acid or the salt thereof with the quantity of N-Acetyl-cysteine.

In one embodiment, reacting the quantity of 2-amino-3-[(2-iodoethoxy)carbonyl]benzoic acid or the salt thereof with the quantity of N-Acetyl-cysteine comprises adding DBU and N-acetyl-cysteine to a stirring solution of 2-amino-3-[(2-iodoethoxy)carbonyl]benzoic acid or a salt thereof in DMF. The DMF may be dry DMF. The method may further comprise stirring the solution. In various embodiments, the solution may be stirred for about 18 hours, under Ar and/or at room temperature. The method may further comprise concentrating the solution to form a concentrated product and/or redissolving the concentrated product in water. The solution may be concentrated under reduced pressure. The redissolved solution may be acidified; for example, with about 37% HCl. The product may be extracted with ethyl acetate. The organic extracts may be washed with water and dried with MgSO₄. The solution may be dried under reduced pressure and redissolved in methanol. Further, the addition of NH₄OH (e.g., 20 μl of 30% NH₄OH) may be performed. Thereafter the product may be crystallized by methanol/ethyl ether.

The present invention also provides for a method of labeling a biomolecule; particularly a biomolecule comprising a sulfhydryl group (also referred to as a thiol group). In one embodiment, the method is for labeling a protein or peptide; particularly amino acid residues with a thiol or sulfhydryl group such as cysteine and lysine or the N-terminus of the protein or peptide. The method comprising provide a fluorescent labeling composition comprising a compound selected from the group consisting of Formula I, Formula II, Formula III, Formula IV, Formula V, Formula VI, salts thereof and combinations thereof and adding the composition to a solution comprising a biomolecule, whereby a biomolecule comprising a sulfhydryl group (also referred to as a thiol group) is labeled. In a particular embodiment, the biomolecule is a protein or peptide. In another particular embodiment, the protein or peptide comprises a cysteine, a lysine or both.

In another embodiment, the method comprises providing a fluorescent labeling composition comprising a compound selected from the group consisting of Compound 1 (I-SHark), Compound 2 (phI-SHark), Compound 3 (I-Requin), Compound 4, Compound 5, Compound 6, salts thereof and combinations thereof, and adding the composition to a solution comprising a biomolecule, whereby a biomolecule comprising a sulfhydryl group or thiol group is labeled. In a particular embodiment, the biomolecule is a protein or peptide. In another particular embodiment, the protein or peptide comprises a cysteine, a lysine or both.

In one embodiment, the quantity of the fluorescent labeling composition may be added in portions. Particularly useful is to add I-Shark or a salt thereof in two or more portions to enhance the solubility of the dye. For example, a portion of the I-SHark or the salt thereof may be added at the beginning of the reaction and a second portion of the I-SHark may be added to the reaction mixture after about 12 hours.

In one embodiment, to label a protein or peptide, the reaction mixture is at a pH of about 7 to about 9. In a particular embodiment, the pH of the solution is at about 7.6. In these conditions, the thiol group on the cysteine residue is allowed to react with the fluorescent labeling composition while other amino acids, such as lysines, are not very reactive with the label. Conditions wherein the pH is below 7 may also be used; however, the reaction tends to slow down and thus labeling may not be as efficient.

In another embodiment, to label a protein or peptide, the solution is at a pH that is greater than 9. In these conditions, the lysine residue and/or the N-terminus of the protein or peptide are quite reactive with the fluorescent labeling composition and become labeled.

In another embodiment, the biomolecule (e.g., protein or peptide) may be purified prior to the labeling procedure. The need for purification of the biomolecules often depends on the application. One of skill in the art will be able to readily determine whether the biomolecule should be purified prior to the labeling procedure.

In embodiments wherein a protein is labeled, the protein may be denatured. Whether to denature the protein depends on the application. In instances wherein the desire is to label all cysteine and/or lysine residues, denaturing the protein prior to the labeling procedure is desirable. However, there are instances wherein it is only desirous to label the surface cysteine or lysine residues of a folded protein and hence, the protein will not be denatured prior to and/or during the labeling procedure.

In another embodiment, the protein may be reduced. Cysteines residues may oxidize, especially cysteine residues on the surface of a folded protein, which result in the formation of a disulfide. The disulfide is not reactive with the fluorescent labeling composition. As such, one may want to place the protein in reduced conditions prior and/or during the labeling procedure.

In another embodiment, the solution comprising the protein, peptide, fluorescent labeling composition may be deoxygenated. Deoxygenation enables the protein or peptide to be reduced and/or remain reduced, which enhances the labeling of the protein or peptide.

In another embodiment, the solution may be oxidized after the protein or peptide has been labeled. This may be beneficial in instances wherein protein complexes may form (e.g., dimers, trimers, etc.) and thus, may increase separation efficiency as the complexes will be larger in size.

The present invention is also directed to a kit for labeling a biomolecule and/or for fluorescence studies, including but not limited to studies on fluorescence energy-transfer kinetics. The kit is an assemblage of materials or components, including at least one of the inventive compositions. Thus, in some embodiments the kit contains a fluorescent composition as described herein.

Instructions for use may be included in the kit. “Instructions for use” typically include a tangible expression describing the technique to be employed in using the components of the kit to effect a desired outcome, such as to use the composition to label a biomolecule or to use the fluorescent composition for fluorescence studies. Optionally, the kit also contains other useful components, such as, diluents, buffers, carriers, cuvettes, applicators, pipetting or measuring tools, other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well known methods, preferably to provide a sterile, contaminant-free environment. The packaging materials employed in the kit are those customarily utilized in fluorescent labeling kits. As used herein, the term “package” refers to a suitable solid matrix or material such as glass, plastic, paper, foil, and the like, capable of holding the individual kit components. Thus, for example, a package can be a glass vial used to contain suitable quantities of an inventive composition as described herein. The packaging material generally has an external label which indicates the contents and/or purpose of the kit and/or its components.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

Unless specified otherwise, all the reagents and solvents were purchased from Sigma and VWR, respectively. Thin Layer Chromatography (TLC) was carried out on silica gel plates (Merck 60-F254). The compounds were visualized using UV light (254 nm and 365 nm). Flash chromatography was carried out using silica gel 60 (EMD Chemicals, 40-63 μm). ¹H- and ¹³C-NMR spectra were collected in CD₃OD with a Varian Mercury 300 MHz spectrometer. Mass analyses were conducted by a ThermoQuest LCQ ion trap mass spectrometer by the California Institute of Technology Protein/Peptide Microanalytical Laboratory. All the elemental analyses were carried out by Desert Analytics, Inc. (Tucson, Ariz.).

### Example 2

### Synthesis of I-SHark

### 2-iodoethyl 2-(methylamino)benzoate (Compound 1)

2-(methylamino)-benzoic acid was first recrystallized from CH₂Cl₂. The recrystallized 2-(methylamino)-benzoic acid (2.55 g, 16.9 mmol), 4-dimethylaminopyridine (DMAP, 320 mg, 2.62 mmol), and 2-iodoethanol (8.56 g, 49.8 mmol) were dissolved in dry CH₂Cl₂ (30 mL). To this mixture, a solution of dicyclohexylcarbodiimide (DCC, 3.80 g, 18.4 mmol) in dry CH₂Cl₂ (10 mL) was added dropwise. The mixture was stirred under Ar at room temperature for 5 h. The solution was filtered through celite, washed with CH₂Cl₂ and dried over Na₂SO₄, and concentrated under vacuum. The concentrated solution was purified under flash column chromatography (hexanes/ethyl acetate 10:1). The purified green oil was then acidified with concentrated HCl. The chloride salt was crystallized from methanol/ether. Yield: 1.56 g, 30%. R_{f}=0.62 (hexanes/ethyl acetate 10:1). ¹H NMR (300 MHz, CD₃OD): δ 8.17 (1H, dd, J=8.1, 1.2 Hz), 7.70 (1H, dt, J=8.4, 1.5 Hz), 7.28-7.34 (2H, m), 4.61 (2H, t, J=6.6 Hz), 3.54 (2H, t, J=6.6 Hz), 3.07 (3H, s). ¹³C NMR (300 MHz, CD₃OD): −0.8, 35.5, 66.1, 119.9, 121.6, 127.2, 132.2, 135.4, 140.7, 165.5. ESI-MS m/z 306 (M+1). Anal. Calcd. for C₁₃ClH₁₃INO₂: C, 35.16; H, 3.84; I, 37.15; N, 4.10. Found: C, 35.23; H, 3.88; I, 37.61; N, 4.11.

### Example 3

### Synthesis of phI-SHark

### 2-amino-3-[(2-iodoethoxy)carbonyl]benzoic acid (Compound 2)

DMAP (27 mg, 0.2 mmol) and 2-iodoethanol (781 mg, 4.45 mmol) were added to a stirring solution of 2-aminoisophthalic acid (274 mg, 1.50 mmol) in THF (20 ml). A solution of DCC (340 mg, 1.65 mmol) was then added dropwise. After stirring for 4 h at room temperature, the mixture was dried under vacuum. The residue was redissolved in diethyl ether and extracted with 5% aqueous NaHCO₃ (3×30 mL). The aqueous layers were combined and acidified with 6M HCl to pH 5-6 and then extracted with ethyl acetate (3×30 mL). Further acidification of the aqueous layer to pH<4 permitted recovery of the starting material, 2-aminoisophthalic acid. The organic phases were combined and dried over MgSO₄, filtered, and concentrated under reduced pressure to yield a pale yellow solid. The crude product was purified by chromatography (diethyl ether/hexanes 3:1), dissolved again in methanol by adding 6M NH₄OH, and crystallized from methanol/methylene chloride. Yield: 48 mg, 14%. R_{f}=0.56 (ethyl acetate/hexanes/acetic acid 3:1:0.01). ¹H NMR (300 MHz, CD₃OD): δ 8.05 (1H, dd, J=7.5, 1.8 Hz), 7.96 (1H, dd, J=7.8, 1.8 Hz), 6.50 (1H, t, J=7.8 Hz), 4.49 (2H, t, J=6.6 Hz), 3.48 (2H, t, J=6.6 Hz). ¹³C NMR (300 MHz, CD₃OD): −0.2, 64.8, 111.0, 112.1, 113.4, 137.2, 138.1, 149.1, 167.1, 176.9. ESI-MS m/z 334 (M−1). Anal. Calcd. for C₁₀H₉INNaO₄: C, 33.64; H, 2.54; I, 35.54; N, 3.92. Found: C, 33.58; H, 2.72; I, 34.97; N, 3.86.

### Example 4

### Synthesis of I-SHark Model Compound

### 2-(acetylamino)-3-[(2-{[2-(methylamino)benzoyl]oxy)ethyl)thio]propanoic acid (Compound 7)

To a stirring solution of I-SHark (305 mg, 1.00 mmol) and N-acetyl-cysteine (326 mg, 2.00 mmol) in dry DMF (20 ml), 1,8-Diazabicyclo[5.4.0]undec-7-ene (DBU, 608 mg, 4.00 mmol) in DMF was added dropwise. The mixture was stirred under Ar for 1 h. The solution was reduced to dryness under vacuum and redissolved in 1M HCl. The solution was then extracted with ethyl acetate (3×30 mL). The organic layers were reduced to dryness under vacuum and redissolved in methanol. After adding 10 μl of 30% NH₄OH solution, the product was crystallized from methanol/ether. Yield: 92 mg, 27%. R_{f}=0.46 (methylene chloride/methanol/acetic acid 4:1:0.1); ¹H NMR (300 MHz, CD₃OD): δ 7.87 (1H, dd, J=8.1, 1.8 Hz), 7.36 (1H, dt, J=8.4, 1.8 Hz), 6.70 (1H, d, J=8.4 Hz), 6.56 (1H, dt, J=8.4, 1.2 Hz), 4.47 (1H, dd, J=7.2, 4.2 Hz), 4.37 (2H, t, J=6.6 Hz), 3.15 (1H, dd, J=13.8, 4.5 Hz), 2.89-2.98 (6H, m), 1.99 (3H, s). ¹³C NMR (300 MHz, CD₃OD): 21.6, 28.4, 30.7, 34.9, 54.5, 63.1, 109.7, 110.6, 114.2, 118.1, 131.5, 134.6, 152.2, 171.6, 175.8. ESI-MS m/z 363 (M+Na). Anal. Calcd. for C₁₅H₂₃N₃O₅S: C, 50.41; H, 6.49; N, 11.76; S, 8.97. Found: C, 49.61; H, 6.24; N, 11.49; S, 8.37.

### Example 5

### Synthesis of phI-SHark Model Compound

### 3-[(2-{[2-(acetylamino)-2-carboxyethyl]thio}ethoxy)carbonyl]-2-aminobenzoic acid (Compound 8)

To a stirring solution of phI-SHark (108 mg, 0.3 mmol) in dry DMF (6 ml), DBU (182 mg, 1.2 mmol) and N-acetyl-cysteine (98 mg, 0.6 mmol) were added. After stirring for 18 h under Ar at room temperature, the mixture was concentrated under reduced pressure and redissolved in water. The solution was acidified with 37% HCl (100 μl) and extracted with ethyl acetate (3×30 mL). The organic extracts were washed with water and dried with MgSO₄. The solution was dried under reduced pressure and redissolved in methanol. After adding 30% NH₄OH (20 μl), the product was crystallized by methanol/ethyl ether. Yield: 32 mg, 32%. R_{f}=0.66 (methylene chloride/methanol/acetic acid 4:1:0.1). ¹H NMR (300 MHz, CD₃OD): δ0 8.10 (1H, dd, J=6.6, 1.8 Hz), 8.07 (1H, dd, J=6.6, 1.8 Hz), 6.57 (1H, t, J=7.8 Hz), 4.51 (1H, dd, J=7.8, 4.5 Hz), 4.42 (2H, t, J=6.6 Hz), 3.16 (1H, dd, J=13.8, 4.5 Hz), 2.90-2.97 (3H, m), 2.00 (3H, s). ESI-MS m/e 393 (M+Na). Anal. Calcd. for C₁₅H₁₇N₂O₇S: C, 46.51; H, 5.46; N, 10.85; S, 8.28. Found: C, 45.51; H, 5.40; N, 9.55; S, 8.31.

### Example 6

### Dye Labeling Procedures

Commercially available cyt c was dissolved in 20 mM NaPᵢ (pH 7.0) buffer. The 100 μM cyt c solution was treated with 1 mM Na₂S₂O₄ and 1 mM DTT. Protein was then purified by ion-exchange chromatography using a Mono S column on a Fast Protein Liquid Chromatography (FPLC) system (Pharmacia). The column was equilibrated with 20 mM NaPᵢ buffer (pH 7.0) and the protein was eluted using a stepwise salt gradient (0-1 M NaCl), with both buffers containing 1 mM dithiothreitol (DTT). Pure fractions were then combined and concentrated to 50-100 μM. Purity of protein was confirmed using absorption and electrospray mass spectrometry.

Purified cyt c (50-100 μM in NaPᵢ, pH 7.0) was exchanged into 20 mM NaPᵢ (pH 7.6) buffer. It was then denatured with 4 M guanidinium chloride (GuHCl) and reduced by the same concentration of Tris(2-carboxy-ethyl)phosphine hydrochloride (TCEP). The reaction mixture was deoxygenated by repeated evacuation/Ar-fill cycles on a Schlenk line for 30 min.

phI-SHark (twenty fold molar excess) was dissolved in the same buffer (1 ml) and added dropwise to the stirring solution of the protein. The reaction was performed in the dark to minimize deleterious photochemical side reactions.

The procedures for I-SHark labeling were slightly modified. Twenty fold molar excess of I-SHark was dissolved in 10% DMSO. The concentrated dye solution was added in two portions (at beginning of the reaction and after 12 h) in order to enhance the solubility of the dye.

The labeling reaction was stirred at room temperature for 40 h. Prior to purification, the solution was centrifuged at 10000×g for 30 min to remove the insoluble dye. The protein was then oxidized with a tenfold excess of K₃[Fe(CN)₆] for 30 min. The reaction mixture was desalted on a gel filtration column (HiPrep Desalting 26/10) using FPLC with 20 mM NaPᵢ (pH 7.0). Dye labeled protein was separated by ion-exchange chromatography (Mono S column) using a shallow stepwise salt gradient (0-1 M NaCl) in 20 mM NaPᵢ (pH 7.0). All the dye-labeled proteins were stored in the dark at 4° C.

The labeling reaction was proven successful by UV-Vis spectroscopy, ESI-MS and SDS-PAGE. Trypsin digestion was also performed with the labeled protein by the California Institute of Technology Protein/Peptide Microanalytical Laboratory. The peptide fragments were then analyzed by MALDI-MS. The results demonstrate that the dye was correctly labeled onto the desired cysteine residue.

### Example 7

### Steady-State Spectroscopy

Spectroscopic measurements for folded protein were recorded at room temperature with samples dissolved in phosphate buffer (20 mM NaPᵢ, pH 7.4), while 6 M guanidinium chloride (GuHCl) was added for the unfolded samples. Samples were deoxygenated by repeated evacuation/Ar-fill cycles during a period for 30 min and sealed in fused-silica fluorescence cuvettes (1 cm×1 cm). Absorption and luminescence spectra were measured on a Hewlett-Packard 8452 diode array spectrophotometer and a Spex Fluorolog2 spectrofluorimeter, respectively. Quantum yields were determined by the method of Demas and Crosby for optically dilute samples (Demas, J. R. (1971) Quantum efficiencies of transition metal complexes 2: Change-transfer luminscence. J. Am. Chem. Soc. 93, 2841). The standard used for quantum yield determination is quinine bisulfate in 1.0 N sulfuric acid, which has an absolute fluorescence quantum yield of 0.55 (Melhuish, W. H. (1961) Quantum efficiencies of fluorescence of organic substances—effect of solvent and concentration of fluorescent solute. J. Phys. Chem. B 65, 229). The excitation wavelength was the same for both samples (355 nm), and the refractive indices for the two solutions were taken to be identical. Reported quantum yields represent averages of multiple measurements at different concentrations (all with OD_{292 nm}<0.1).

### Example 8

### Evaluation of the Förster Distance

The value of overlap integral (J, equation 2) for the donor-acceptor pairs (I-SHark/heme and phI-SHark/heme) was determined using the absorption spectrum for cyt c and normalized emission spectra of compounds 2-(acetylamino)-3-[(2-{[2-(methylamino)benzoyl]oxy}ethyl)thio]propanoic acid and 3-[(2-{[2-(acetylamino)-2-carboxyethyl]thio}ethoxy)carbonyl]-2-aminobenzoic acid. The solution refractive index (n) was taken to be 1.34 (Thormahlen et al. (1985) Refractive index of water and its dependence on wavelength, temperature and density. J. Phys. Chem. Ref Data 14, 933-946), while the orientation factor (κ²) was averaged to be ⅔ for the random dynamic orientation of the donor and acceptor.

J=∫F_{D}(λ)ε_{A}(λ)λ⁴dλ (Equation 2)

### Example 9

### Time-Resolved Fluorescence Spectroscopy

Samples for time-resolved fluorescence spectroscopy measurements were prepared as described above. Samples were excited by a 355-nm polarized pulse (35° from vertical) generated from a mode-locked Nd:YAG laser regenerative amplifier. Emission above 420 nm was selected by cutoff filters. Decay kinetics were measured with a picosecond streak camera (Hamamatsu C5680) in single photon counting mode. All experiments were controlled by a temperature-controlled cuvette holder at 25° C. Control experiments were conducted using the model compounds 2-(acetylamino)-3-[(2-{[2-(methylamino)benzoyl]oxy}ethyl)thio]propanoic acid and 3-[(2-{[2-(acetylamino)-2-carboxyethyl]thio}ethoxy)carbonyl]-2-aminobenzoic acid.

### Example 10

### Data Analysis

The measured fluorescence decay kinetics were fitted by previously described procedures (Lee et al. (2007) a-Synuclein tertiary contact dynamics. J. Phys. Chem. B 111, 2107-2112). Kinetic traces were modeled with the function I(t)=Σₖ P(k)exp(−kt), where I(t) is the fluorescence decay and P(k) is the probability distribution of excited state decay rate constants. The critical constraint for this inversion is the requirement that P(r)≧0. A linear least-squares (LLS) MATLAB (Mathworks, Natick, Mass.) algorithm with a nonnegativity constraint (LSQNONNEG) was used to project the narrowest P(k) distributions from the fluorescence kinetics (Lee et al. (2004) α-synuclein structures from fluorescence energy-transfer kinetics: Implications for the role of the protein in Parkinson's disease. Proc. Natl. Acad. Sci. 101, 16466-16471). This method of data analysis is referred to as non-negative linear least-squares (NNLS).

### Example 11

### Synthesis of I-Requin (Compound 3)

Condensation of an amino group on a dye precursor (e.g., 2-aminobenzoic acid) with a halo-acetyl chloride reagent (e.g., iodoacetyl chloride, commercially available) was performed. The condensation reaction was run in mildly basic aqueous solution to ensure deprotonation of the amine group. Upon completion of the reaction, the solution was acidified and the product (I-Requin) precipitates.

### Example 12

### Synthesis of I-Requin's Derivatives (Compounds 4-6)

Reactions involve condensation of an amino group on a dye precursor with a halo-acetyl chloride reagent (e.g., chloroacetyl chloride, iodoacetyl chloride; both commercially available). The condensation reaction is run in mildly basic aqueous solution to insure deprotonation of the amine group. Upon completion of the reaction, the solution is acidified and the product (I-Requin derivatives) precipitates.

While the description above refers to particular embodiments of the present invention, it should be readily apparent to people of ordinary skill in the art that a number of modifications may be made without departing from the spirit thereof. The accompanying claims are intended to cover such modifications as would fall within the true spirit and scope of the invention. The presently disclosed embodiments are, therefore, to be considered in all respects as illustrative and not restrictive, the scope of the invention being indicated by the appended claims rather than the foregoing description. All changes that come within the meaning of and range of equivalency of the claims are intended to be embraced therein.

What is claimed is:

1. A composition, comprising a compound selected from the group consisting of: 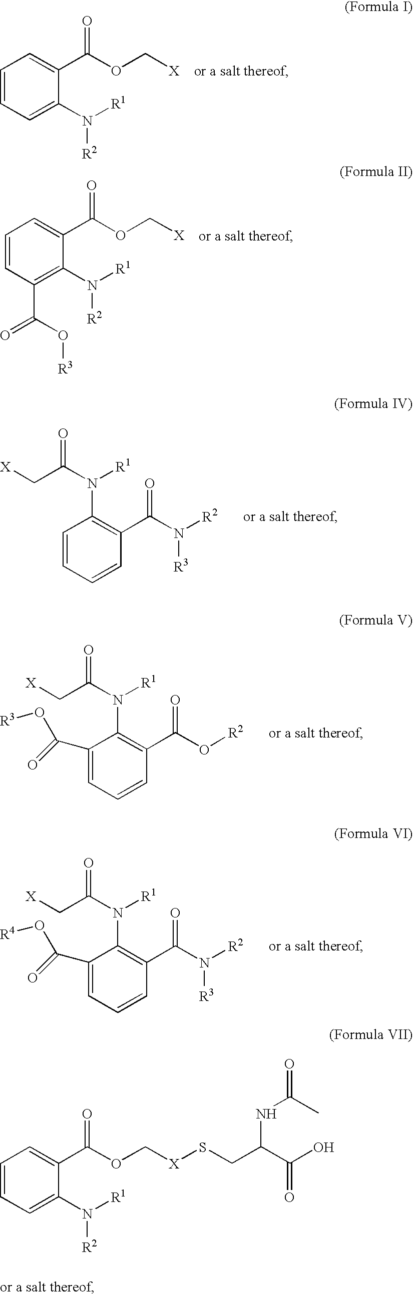 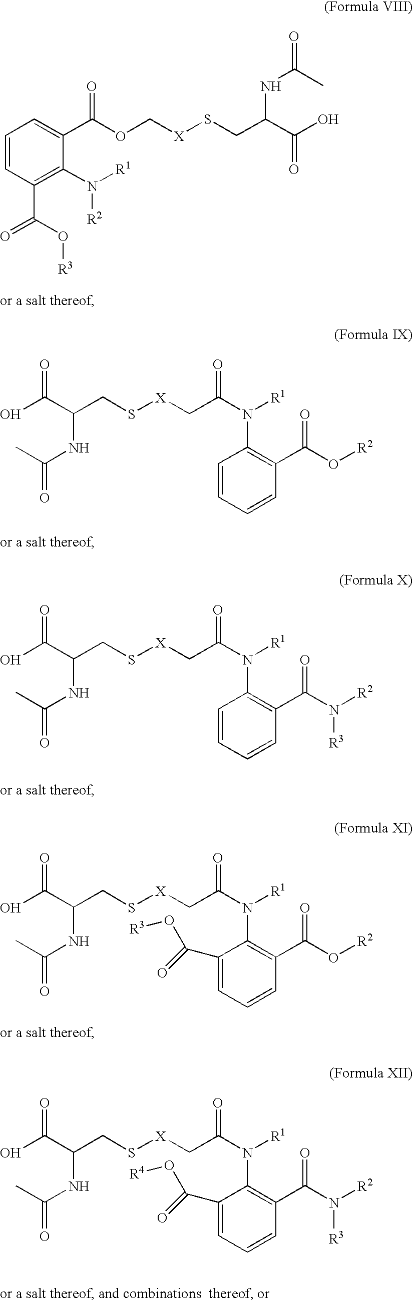 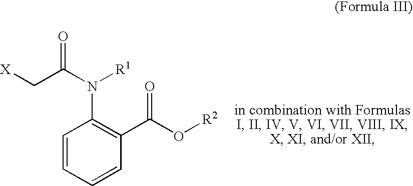
wherein R¹, R², R³ and R⁴ are each independently selected from hydrogen or functional groups C₂-C₁₈ alkenyl, C₂-C₁₈ alkynyl, C₁-C₁₈ alkyl, aryl, C₁-C₁₈ carboxylate, C₂-C₁₈ alkoxy, C₂-C₁₈ alkenyloxy, C₂-C₁₈ alkynyloxy, aryloxy, C₂-C₁₈ alkoxycarbonyl, C₁-C₁₈ alkylthio, C₁-C₁₈ alkylsulfonyl or C₁-C₁₈ alkylsulfinyl,
each functional group is optionally substituted with C₁-C₅ alkyl, a halogen, C₁-C₅ alkoxy or with a phenyl group optionally substituted with a halogen, C₁-C₅ alkyl or C₁-C₅ alkoxy; and
X is selected from F, Cl, Br, I, —SO₃X, a carboxylic acid, a salt of carboxylic acid, CN, nitro, hydroxy, azido, amino, and hydrazino, or X is selected from C₁-C₁₈ alkyl, C₁-C₁₈ alkoxy, C₁-C₁₈ alkylthio, C₁-C₁₈ alkanoylamino, C₁-C₁₈ alkylaminocarbonyl, C₂-C₃₆ dialkylaminocarbonyl, C₁-C₁₈ alkyloxycarbonyl, or C₆-C₁₈ arylcarboxamido, the alkyl or aryl portions optionally substituted one or more times by F, Cl, Br, I, hydroxy, carboxylic acid, a salt of carboxylic acid, a carboxylic acid ester of a C₁-C₆ alcohol, —SO₃X, amino, alkylamino, dialkylamino and alkoxy, the alkyl portions of these substituents in turn having 1-6 carbons.

2. The composition of claim 1, wherein the compound is selected from the group consisting of:

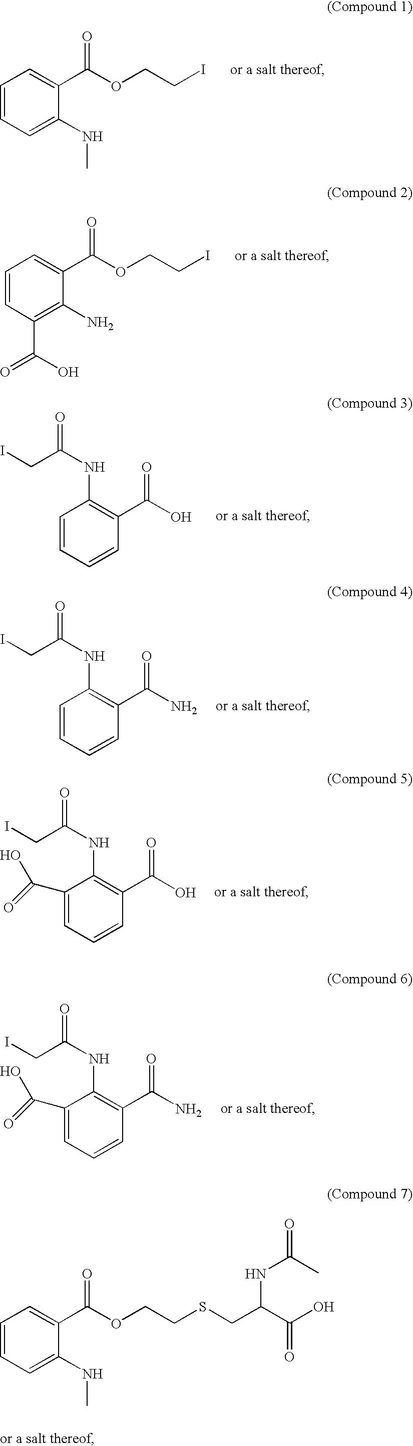

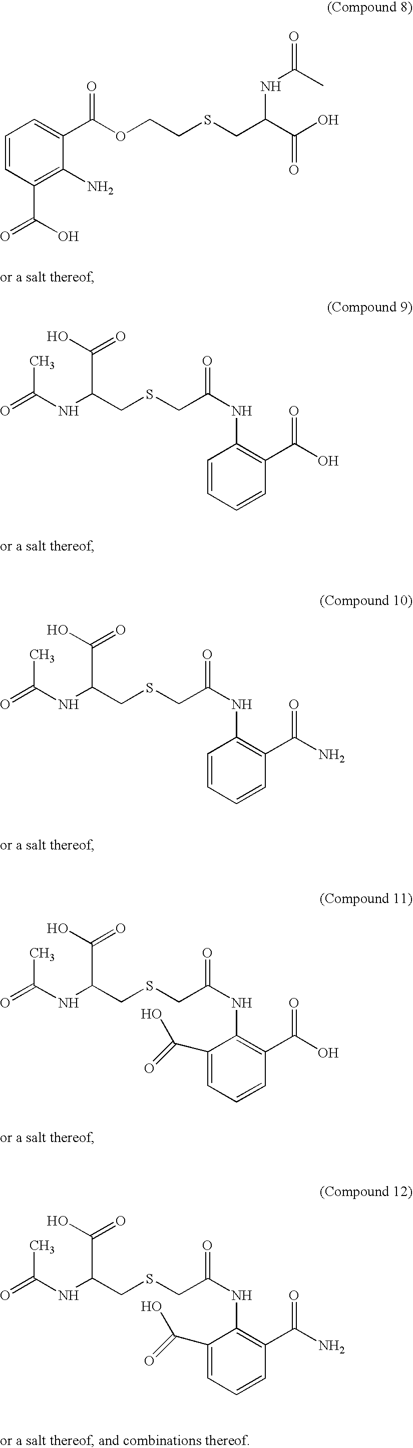

3. The composition of claim 2, wherein the compound is Compound 1 or a salt thereof.

4. The composition of claim 2, wherein the compound is Compound 2 or a salt thereof.

5. The composition of claim 2, wherein the compound is Compound 4 or a salt thereof.

6. The composition of claim 2, wherein the compound is Compound 5 or a salt thereof.

7. The composition of claim 2, wherein the compound is Compound 6 or a salt thereof.

8. The composition of claim 2, wherein the compound is Compound 7 or a salt thereof.

9. The composition of claim 2, wherein the compound is Compound 8 or a salt thereof.

10. The composition of claim 2, wherein the compound is Compound 9 or a salt thereof.

11. The composition of claim 2, wherein the compound is Compound 10 or a salt thereof.

12. The composition of claim 2, wherein the compound is Compound 11 or a salt thereof.

13. The composition of claim 2, wherein the compound is Compound 12 or a salt thereof.

14. The composition of claim 3, produced by the process comprising:
providing a quantity of 2-(methylamino)-benzoic acid and a quantity of 2-iodoethanol; and
reacting the quantity of 2-(methylamino)-benzoic acid and the quantity of 2-iodoethanol in a reaction mixture,
wherein the reacting is optionally performed in the presence of a quantity of 4-dimethylaminopyridine (“DMAP”) and a quantity of dicyclohexylcarbodiimide (“DCC”), the quantities of 2-(methylamino)-benzoic acid, 2-iodoethanol and DMAP are optionally dissolved in methylene chloride, and the quantity of DCC is optionally added dropwise to the reaction mixture.

15. The composition of claim 4, produced by the process comprising:
providing a quantity of 2-aminoisophthalic acid and a quantity of 2-iodoethanol; and
reacting the quantity of 2-aminoisophthalic acid and the quantity of 2-iodoethanol in a reaction mixture,
wherein the reacting is optionally performed by adding a quantity of 4-dimethylaminopyridine (“DMAP”) and the quantity of 2-iodoethanol to a stirring solution of 2-aminoisophthalic acid in tetrahydrofuran (“THF”), and adding a quantity of dicyclohexylcarbodiimide (“DCC”), and the quantity of DCC is optionally added dropwise.

16. The composition of claim 1, wherein the compound is 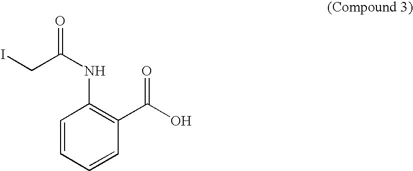
or a salt thereof and is produced by the process comprising:
providing a quantity of iodoacetyl chloride and a quantity of 2-aminobenzoic acid; and
reacting quantities of iodoacetyl chloride and 2-aminobenzoic acid by condensation.

17. The composition of claim 1, further comprising a biomolecule.

18. The composition of claim 17, wherein the biomolecule is a protein, peptide, or both.

19. A method of labeling a biomolecule, comprising:
providing a composition comprising a compound selected from the group consisting of: 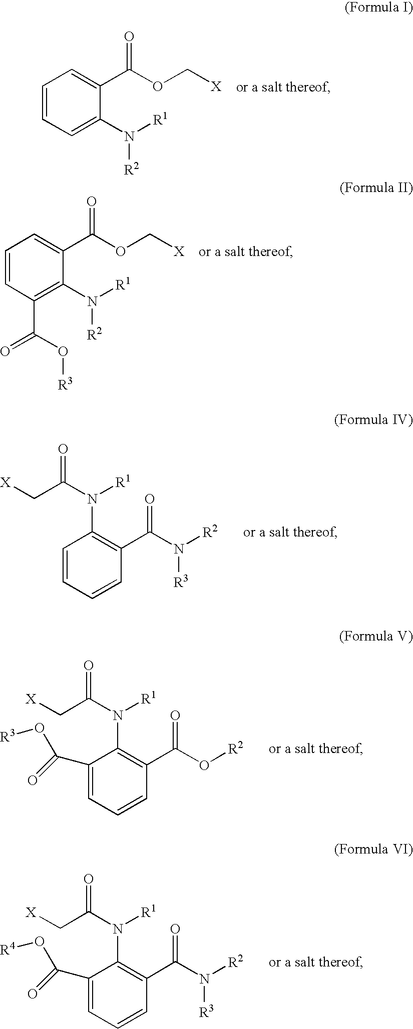
combinations thereof,
wherein R¹, R², R³ and R⁴ are each independently selected from hydrogen or functional groups C₂-C₁₈ alkenyl, C₂-C₁₈ alkynyl, C₁-C₁₈ alkyl, aryl, C₁-C₁₈ carboxylate, C₂-C₁₈ alkoxy, C₂-C₁₈ alkenyloxy, C₂-C₁₈ alkynyloxy, aryloxy, C₂-C₁₈ alkoxycarbonyl, C₁-C₁₈ alkylthio, C₁-C₁₈ alkylsulfonyl or C₁-C₁₈ alkylsulfinyl,
each functional group is optionally substituted with C₁-C₅ alkyl, a halogen, C₁-C₅ alkoxy or with a phenyl group optionally substituted with a halogen, C₁-C₅ alkyl or C₁-C₅ alkoxy; and
X is selected from F, Cl, Br, I, —SO₃X, a carboxylic acid, a salt of carboxylic acid, CN, nitro, hydroxy, azido, amino, and hydrazino, or X is selected from C₁-C₁₈ alkyl, C₁-C₁₈ alkoxy, C₁-C₁₈ alkylthio, C₁-C₁₈ alkanoylamino, C₁-C₁₈ alkylaminocarbonyl, C₂-C₃₆ dialkylaminocarbonyl, C₁-C₁₈ alkyloxycarbonyl, or C₆-C₁₈ arylcarboxamido, the alkyl or aryl portions optionally substituted one or more times by F, Cl, Br, I, hydroxy, carboxylic acid, a salt of carboxylic acid, a carboxylic acid ester of a C₁-C₆ alcohol, —SO₃X, amino, alkylamino, dialkylamino and alkoxy, the alkyl portions of these substituents in turn having 1-6 carbons; and
adding the composition to a mixture comprising a biomolecule to create a reaction mixture,
whereby a biomolecule comprising a sulfhydryl group is labeled.

20. The method of claim 19, wherein the compound is selected from the group consisting of:

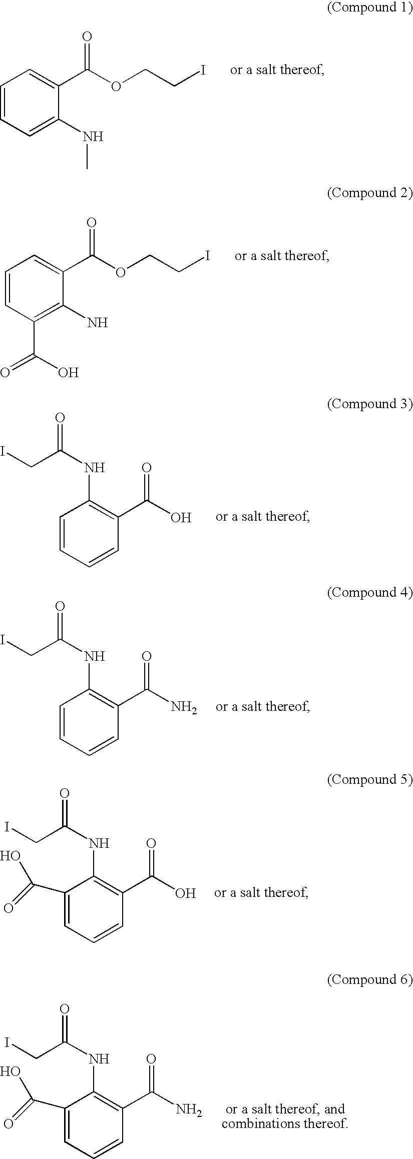

21. The method of claim 19, wherein the biomolecule is a protein or a peptide.

22. The method of claim 21, wherein the protein or the peptide comprises a cysteine, a lysine or both.

23. The method of claim 19, wherein the composition is added to the mixture in portions.

24. The method of claim 19, wherein the reaction mixture is at a pH of about 7 to about 9.

25. The method of claim 24, wherein the reaction mixture is at a pH of about 7.6.

26. The method of claim 19, wherein the reaction mixture is at a pH of 9 or greater.

27. A method of producing a composition, comprising:
providing a quantity of 2-(methylamino)-benzoic acid and a quantity of 2-iodoethanol; and
reacting the quantity of 2-(methylamino)-benzoic acid and the quantity of 2-iodoethanol in a reaction mixture,
wherein the reacting is optionally performed in the presence of a quantity of 4-dimethylaminopyridine (“DMAP”) and a quantity of dicyclohexylcarbodiimide (“DCC”), the quantities of 2-(methylamino)-benzoic acid, 2-iodoethanol and DMAP are optionally dissolved in methylene chloride, and the quantity of DCC is optionally added dropwise to the reaction mixture, and
whereby a compound with the formula

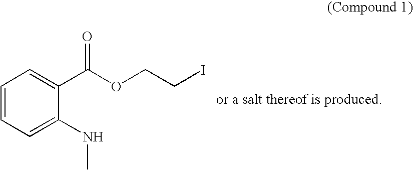

28. A method of producing a composition, comprising:
providing a quantity of 2-aminoisophthalic acid and a quantity of 2-iodoethanol; and
reacting the quantity of 2-aminoisophthalic acid and the quantity of 2-iodoethanol in a reaction mixture,
wherein the reacting is optionally performed by adding a quantity of 4-dimethylaminopyridine (“DMAP”) and the quantity of 2-iodoethanol to a stirring solution of 2-aminoisophthalic acid in tetrahydrofuran (“THF”), and adding a quantity of dicyclohexylcarbodiimide (“DCC”), and the quantity of DCC is optionally added dropwise, and
whereby a compound with the formula

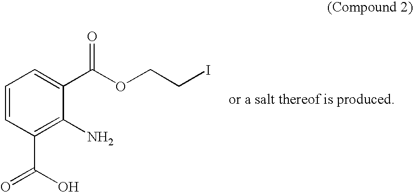

29. A kit for labeling a biomolecule or for fluorescence studies, comprising:
a composition, comprising a compound selected from the group consisting of: 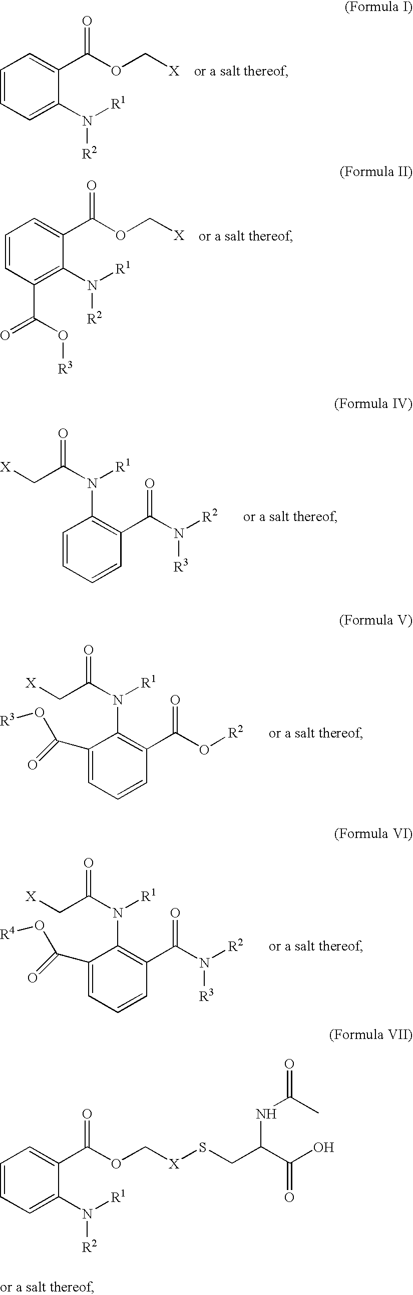 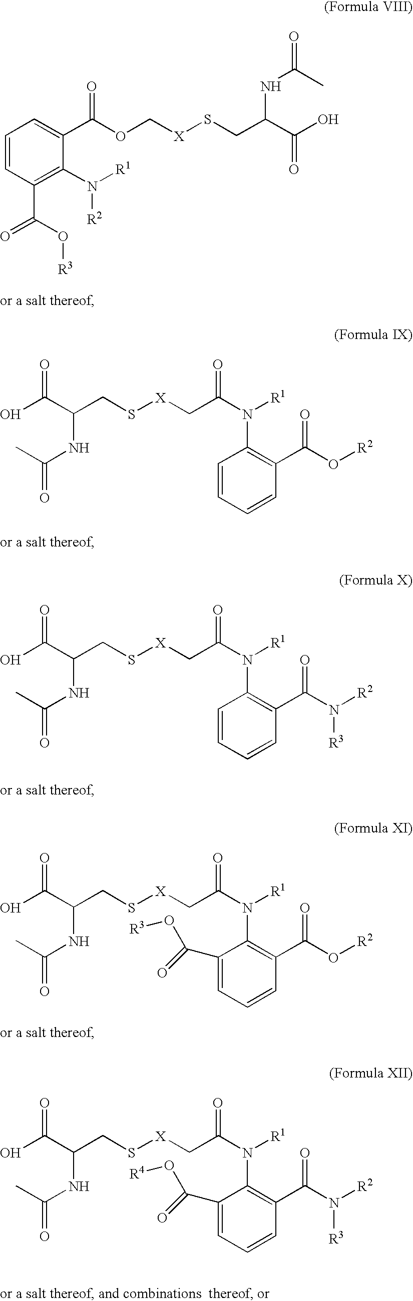 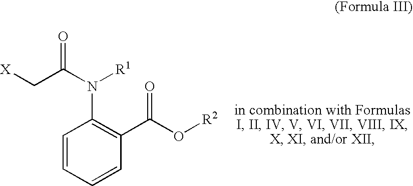
wherein R¹, R², R³ and R⁴ are each independently selected from hydrogen or functional groups C₂-C₁₈ alkenyl, C₂-C₁₈ alkynyl, C₁-C₁₈ alkyl, aryl, C₁-C₁₈ carboxylate, C₂-C₁₈ alkoxy, C₂-C₁₈ alkenyloxy, C₂-C₁₈ alkynyloxy, aryloxy, C₂-C₁₈ alkoxycarbonyl, C₁-C₁₈ alkylthio, C₁-C₁₈ alkylsulfonyl or C₁-C₁₈ alkylsulfinyl,
each functional group is optionally substituted with C₁-C₅ alkyl, a halogen, C₁-C₅ alkoxy or with a phenyl group optionally substituted with a halogen, C₁-C₅ alkyl or C₁-C₅ alkoxy; and
X is selected from F, Cl, Br, I, —SO₃X, a carboxylic acid, a salt of carboxylic acid, CN, nitro, hydroxy, azido, amino, and hydrazino, or X is selected from C₁-C₁₈ alkyl, C₁-C₁₈ alkoxy, C₁-C₁₈ alkylthio, C₁-C₁₈ alkanoylamino, C₁-C₁₈ alkylaminocarbonyl, C₂-C₃₆ dialkylaminocarbonyl, C₁-C₁₈ alkyloxycarbonyl, or C₆-C₁₈ arylcarboxamido, the alkyl or aryl portions optionally substituted one or more times by F, Cl, Br, I, hydroxy, carboxylic acid, a salt of carboxylic acid, a carboxylic acid ester of a C₁-C₆ alcohol, —SO₃X, amino, alkylamino, dialkylamino and alkoxy, the alkyl portions of these substituents in turn having 1-6 carbons; and
instructions to use the composition to label the biomolecule, or
instructions to use the composition for fluorescence studies.

30. The kit of claim 29, wherein the compound is selected from the group consisting of: 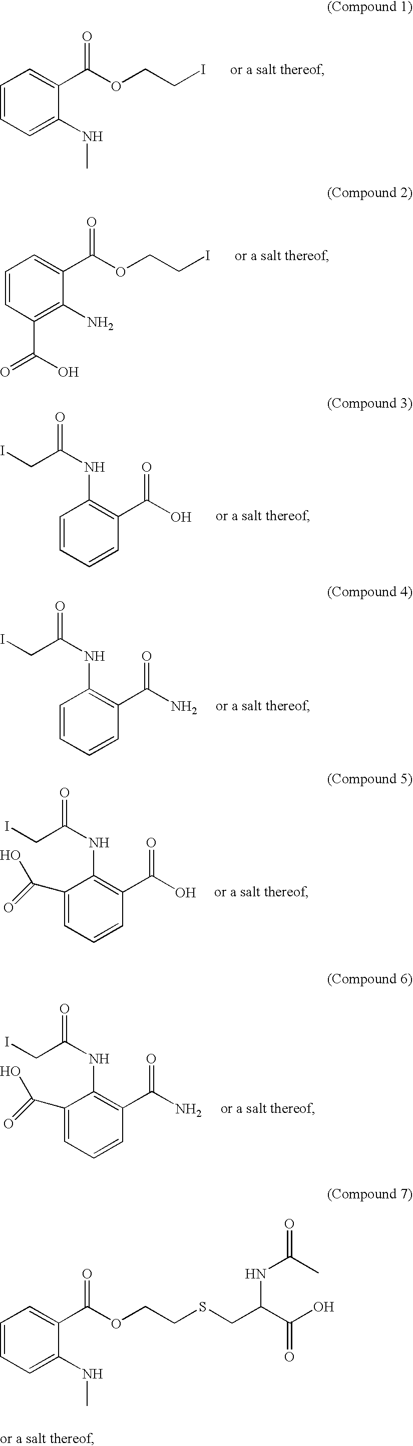 [chemical structure]
